(19) United States
(12) Patent Application Publication
(10) Pub. No.: US 2021/0068425 A1
(43) Pub. Date: Mar. 11, 2021

(54) PALATABLE GRANULAR VETERINARY COMPOSITIONS

(71) Applicant: Bayer Healthcare LLC, Whippany, NJ (US)

(72) Inventors: Stacy ROSS, Lisbon, IA (US); Jared STEFANICK, Greenfield, IN (US)

(21) Appl. No.: 17/012,777

(22) Filed: Sep. 4, 2020

Related U.S. Application Data
(60) Provisional application No. 62/897,103, filed on Sep. 6, 2019.

(51) Int. Cl. A23K 50/42; A23K 40/10; A23K 10/30; A23K 20/105; A23K 20/163; A23K 50/48; A23K 40/25

(52) U.S. Cl. CPC A23K 50/42; A23K 40/10; A23K 10/30; A61K 31/155; A23K 20/163; A23K 50/48; A23K 40/25; A23K 20/105

(57) ABSTRACT

The present invention provides palatable granule veterinary compositions comprising at least one active pharmaceutical ingredient, at least one wetting agent, and at least one palatant. The present invention also provides methods for controlling or treating a condition in an animal comprising administering the palatable composition to said animal in need thereof.

This application claims priority from U.S. provisional application Ser. No. 62/897,103, filed Sep. 6, 2019.

The present invention is directed to a palatable granular veterinary composition comprising at least one active agent, at least one wetting agent, and at least one palatant or flavorant, and methods for controlling or treating a condition in an animal comprising administering the composition to said animal in need thereof.

### BACKGROUND

There is an ongoing need to develop effective, highly palatable dosage forms for delivery of active veterinary ingredients to animals.

The ease of administering oral veterinary medication to an animal is a major aspect of owner compliance and has a significant impact on an animal's health. An animal's willingness to voluntarily ingest medicine is dependent upon the palatability of the dosage form.

When an owner or trainer places veterinary medicine in a feeding bowl or other receptacle, or in an outstretched hand, it is incumbent that animals willingly and by free choice accept and consume the medicine. However, most oral medications have a bitter taste and/or an offensive aroma to animals, which renders medicating animals difficult.

Palatability of a veterinary dosage form is determined by the smell, taste and feeling of the medicine in the mouth (commonly referred to as “good mouth feel”). In general, palatability is attained by adding a palatant to a formulation during the manufacturing process.

Some palatable, chewable dosage forms are designed to be voluntarily consumed by animals, such as chewable tablets (i.e., “hard-chews compositions” or “hard chews”) and soft-chew compositions (i.e., “soft chews”).

However, when an animal is non-compliant or otherwise unwilling to receive a veterinary dosage form, animal owners and trainers generally administer oral medications via one of four methods.

First, owners and trainers may inject a liquid oral medication directly into an animal's throat. Secondly, owners and trainers may apply oral medication in liquid drops to the animal's food. Thirdly, owners and trainers may administer oral medication in liquid drops to the animal orally.

Finally, owners and trainers may employ the ‘poke down’ method. If the animal has lost its appetite or the medicine cannot be given with food, the owner or trainer will have the unpleasant task of poking a solid dosage form (e.g., a tablet, soft chew, or capsule) down the animal's throat. Owners and trainers may find it easier to keep a large dog, for example from wriggling away by straddling it and holding its shoulders steady between their knee while making sure not to put weight on the dog's back. The owner or trainer must, with one hand, grasp over the top of the animal's muzzle and carefully pull the bottom jaw down with the opposite hand. Very quickly, the owner or trainer must poke the tablet or capsule as far back in the animal's throat as possible and close the mouth, firmly holding it shut with the one hand, while gently stroking the throat with the opposite hand, until the animal swallows.

Each of these methods requires coercion, force, and/or trickery. If an animal is not hungry or is particularly resistant, compliance, and therefore treatment success, will be significantly diminished. These methods are highly challenging for owners, especially if medicine is needed to be given on an empty stomach or if long-term medication is required. Accordingly, chewable solid, palatable dosage forms are preferable.

Common chewable solid dosage forms include hard-chew compressed tablets, which generally comprise palatants and coatings to improve palatability. However, dosage form texture must also be considered during manufacturing. Hard-chew compressed tablets, for example, tend to be gritty or otherwise unappealing to animals. Generally, animal owners and trainers must still employ the ‘poke down’ method with hard chews or resort to hiding hard chews in other food or treats, despite the fact that they are marketed as chewable dosage forms.

Further still, soft-chew compositions may be administered, which may or may not comprise one or more palatants.

There is a need for improved formulations of a veterinary active agent into a desirable edible medication to increase an animal's voluntary acceptance of veterinary medication. There is also a need for additional palatable dosage forms which are voluntarily consumed by subject animals.

### SUMMARY

The present inventors have found that palatable granular compositions described herein exhibit high palatability and, as a result thereof, high animal acceptance and owner compliance.

The present invention provides for palatable granular compositions comprising

(a) at least one palatant;

(b) at least one wetting agent;

(c) at least one active ingredient;

The disclosure further provides for methods of treating animals with diseases or conditions, comprising administering a palatable granular composition of the present disclosure to said animal.

Further objects, features, and advantages of the invention will become apparent from the detailed description that follows.

### DETAILED DESCRIPTION

Applicants have now found that palatable granular dosage forms of the present invention demonstrate superior acceptance among animals.

Palatable granular compositions of the present disclosure maximize the use of palatable components, rather than typical pharmaceutical ingredients, to achieve high palatability.

Typical pharmaceutical ingredients may not taste or smell appealing to animals, which can result in poor compliance.

Accordingly, palatable granular compositions of the present disclosure can achieve high drug loads and produce superior pharmaceutical effect in treated animal subjects.

Marketed veterinary products generally require at least 17 minutes to disintegrate, and in many cases, more than 60 minutes. Improved disintegration time allows for absorption of a variety of active pharmaceutical ingredients across the gastrointestinal system and may prevent the complaint of dosage forms passing through an animal subject intact.

It is common in the field for granules to be included in a capsule, a tablet (i.e., hard-chew composition) or soft-chew composition for the final dosage form.

In general, veterinary granules are much smaller than the dosage forms into which they are commonly incorporated, such as capsules, tablets and soft-chew compositions. For example, veterinary granules may be, generally, nanometer sizes, micrometer sizes, or agglomerates of nanometer-sized granules to form micron-size granules. In general, several granules are required to make up the same mass as a chewable tablet or soft chew mass.

The palatable granules of the present invention, however, are a final dosage form and can be voluntarily consumed by animals.

The palatable granular compositions of the present invention have the unique advantage of being, essentially, already disintegrated when compared to an intact soft-chew dosage form.

The palatable granule compositions of the present invention represent an improvement over existing granule formulations, which are incorporated merely as components of a further final dosage form. However, the palatable granule compositions of the present invention may also be incorporated into a capsule, tablet, or soft chew dosage form, as it traditionally done.

In general, the palatable granules of the present invention are smaller than chewable tablets (i.e., hard-chew compositions) or soft-chew compositions. It would take multiple granules to have the same mass as a chewable tablet or soft chew. Thus, a further advantage of the palatable granules of the present invention is that the dosage of active ingredient to be administered to an animal can be easily adjusted by administering additional palatable granules to be voluntarily consumed by the animal.

Thus, the palatable granular compositions of the present invention represent a wholly unique veterinary dosage form not previously available to animal owners, trainers, or veterinarians.

“Animal” means an individual animal belonging to the class Mammalia, Reptilia or Aves. In an aspect, palatable granular compositions of the present invention may be administered to an animal.

In another aspect, palatable granular compositions of the present invention may be administered to a mammal or a bird.

In another aspect, palatable granular compositions of the present invention may be administered to animals such as dogs, cats, horses, pigs, llamas, rabbits, goats, sheep, deer, elk, cattle and poultry.

“Subject” means an animal to which a palatable granule of the present invention is administered for treatment, prevention, and/or amelioration of a disease or condition and/or symptoms thereof.

“Granule composition” or “granule dosage form” or “palatable granule” or “palatable granule composition” means a dosage form which an animal is capable of chewing or swallowing whole and ingesting. A palatable granule composition of the present invention is generally smaller than a soft-chew veterinary composition. In general, multiple palatable granules of the present invention would be required to make up the mass of a typical soft-chew composition. Palatable granules of the present invention may be manufactured by, for example, wet granulation and dry granulation methods, including spray drying, fluid bed, high shear wet granulation, low shear wet granulation, dry granulation in a ribbon blender, mortar and pestle, and other known methods of manufacture.

Granules are an efficient way to incorporate active pharmaceutical ingredients with other ingredients. Typically, if one or more active ingredients is/are incorporated into a granule, the granule is further incorporated into other final dosage forms, such as pet food, gels, capsules, tablets, and soft or hard chewables.

The present inventors have found that palatable granule compositions of the present invention, however, are voluntarily consumed by subject animals even when not incorporated into a further final dosage form. Rather, the palatable granules of the present invention represent a final dosage form in themselves and need not be incorporated into a tablet or chew, or be mixed with pet food. The present invention represents an improvement over existing granule compositions which are not themselves palatable and are, for example, sprinkled on top of, or otherwise mixed with, pet foods or livestock feed. By contrast, palatable granules of the present invention are voluntarily consumed by an animal to be treated as though the palatable granules were themselves food.

Palatable granule compositions of the present invention have the distinct advantage over chewable compositions of being capable of high drug loading, as demonstrated by the Examples below.

Thus, in certain embodiments, palatable granule compositions of the present invention may be employed as a final dosage form.

In other embodiments, palatable granule compositions of the present invention may be incorporated into further dosage forms, including pet foods, gels, capsules, tablets, soft chews and/or hard chews.

In an aspect, palatable granule compositions of the present invention do not need to be mixed or otherwise combined with other palatable materials, such as pet food.

In another aspect, palatable granule compositions of the present invention may be mixed or otherwise combined with other palatable materials, such as pet food, if desired.

For use in the invention, no inactive ingredients of the palatable granules of the present invention should be of less than food grade quality and may be of higher quality (e.g., USP or NF grade). In this context, “food grade” means that the material does not contain or impart chemicals or agents hazardous to health. Thus, a food grade flavoring, if of animal origin, will be one that has been prepared to substantially reduce or eliminate the presence of infectious agents or contaminants therein; e.g., by processes such as pasteurization, pressurization or irradiation. The latter process, in particular, can effectively eliminate infectious agents such as E. coli, Salmonella and Campylobacter from a wide variety of food and animal-derived substances, such as raw meat products, vegetables, grains and fruits.

In certain embodiments, palatable granules of the invention will not contain any animal origin ingredients, and/or will not contain any animal origin flavorings.

In other embodiments, palatable granules of the invention may contain ingredients, e.g. flavorants, of animal origin.

All ingredients should be pharmaceutically acceptable (e.g., food grade, USP or NF, as appropriate).

“Pharmaceutically acceptable” means that an ingredient, substance or composition must be compatible chemically and/or toxicologically, with the other ingredients within a formulation, composition, and/or the animal being treated therewith.

“Palatant” means a non-active flavoring ingredient that entices a pet to consume a food, treat, supplement or veterinary medicine. Palatants to be used in compositions of the present invention may take the form of dry powder palatants, non-powder palatants, or as systems that use both dry powder and non-powder palatants.

In an aspect, palatable granule compositions of the present invention comprise dry powder palatants. Suitable palatable powders include plant- and animal-derived flavoring agents and artificial meat flavorings. In an aspect, compositions of the present invention comprise palatants derived from fruits, vegetables, beef, poultry, fish and/or artificial meat flavorings.

In an aspect, palatable granule compositions of the present invention comprise one or more palatable powders selected from sugar, sugar substitutes, salt, bone marrow, blood meal, by-product meal, aroma powders or liquids, apple powder, bean powder, beet powder, pepper powder, blueberry powder, broccoli powder, squash powder, cabbage powder, carrot powder, cauliflower powder, celery powder, chevril powder, chive powder, corn powder, cranberry powder, dill powder, kale powder, leek powder, lemon powder, mushroom powder, onion powder, orange powder, potato powder, pea powder, pumpkin powder, shallot powder, spinach powder, tomato powder, tomatillo powder, sweet potato powder, zucchini powder, other vegetable or fruit powders, and/or natural and artificial meat powders and other solid meat palatants, including liver and beef, as well as commercially available palatants.

In another aspect, palatable granules compositions of the present invention comprise a palatant selected from blueberry powder, carrot powder, sweet potato powder, liver powder, and/or artificial beef.

In another aspect, a palatant to be used in a palatable granule composition of the present invention may alternatively be a chip or other solid palatant, rather than a powder.

In an aspect, palatable granule compositions of the present invention comprise one or more non-powder palatants, such as yeast, yeast extract, tapioca syrup, honey, and/or salt.

In an aspect, palatable granule compositions of the present invention comprise one or more palatants in a total amount of 1% to 90%, or 10% to 80%, or 20% to 70%, or 30% to 60%, by weight based on the total weight of the palatable granule composition.

In an aspect, palatable granule compositions of the present invention may comprise salt and/or sugar, which are known to be highly palatable to dogs.

“Pharmaceutically effective amount” means a nontoxic amount of the active ingredient that is sufficient to effect beneficial or desired results as described herein when administered to a subject. Effective administration—i.e., feeding a palatable granule composition to a subject animal—and dosage amounts may be determined empirically, and making such determinations is within the skill of the art. It is understood by those skilled in the art that the dosage amount will vary with the rate of excretion, the duration of the treatment, the identity of any other drugs being administered, the age, size, and species of animal and like factors well known in the art of veterinary medicine. In general, a suitable dose of the composition according to the invention will be that amount of the composition, which is the lowest dose effective to produce the desired effect with no or minimal side effects.

The amount of active ingredient depends on the active ingredient, the animal being treated, the state of the animal's condition, and the severity of the condition. The determination of those factors is well within the level of one skilled in the veterinary arts.

“Active ingredient” should be understood in its normal sense and covers ingredients pharmaceutically acceptable and effective for treatment of the animal body as well as an association of one or several such medicaments. In an aspect, palatable granule compositions of the present invention may comprise any active ingredient suitable for oral ingestion. In an aspect, the palatable granule compositions of the present invention comprise at least one active ingredient may include agents that are, for example, antiparasitic (endo- or ecto-), acaricidic, anthelmintic, insecticidal, antimicrobial, antiviral, antibiotic, anti-inflammatory, psychotropic, proton pump inhibitors, pain-relieving, anti-allergy, antihypertensive, and any other active ingredient useful in treating animal conditions.

The active ingredient can be, for example, one or more acaricides selected from the group of acaricide classes consisting of antibiotic acaricides such as abamectin, doramectin, enamectin, eprinomectin, ivermectin, lepimectin, milbemectin, nikkomycins, selamectin, tetranactin, and thuringiensin; bridged diphenyl acaricides such as azobenzene, benzoximate, benzyl benzoate, bromopropylate, chlorbenside, chlorfenethol, chlorfenson, chlorfensulphide, chlorobenzilate, chloropropylate, dicofol, diphenyl sulfone, dofenapyn, fenson, fentrifanil, fluorbenside, proclonol, tetrad ifon, and tetrasul; carbamate acaricides such as benomyl, carbanolate, carbaryl, carbofuran, fenothiocarb, methiocarb, metoicarb, promacyi, and propoxur; oxime carbamate acaricides such as aldicarb, butocarboxim, oxamyl, thiocarboxime, and thiofanox; dinitrophenol acaricides such as binapacryl, dinex, dinobuton, dinocap, dinocap-4, dinocap-6, dinocton, dinopenton, dinosulfon, dinoterbon, and DNOC; formamidine acaricides such as amitraz, chlordimeform, chloromebuform, formetanate, and formparanate, mite growth regulators such as cbfentezine, dofenapyn, fluazuron, flubenzimine, flucycloxuron, flufenoxuron, and hexythia-zox; organochlorine acaricides such as bromocyclen, camphechlor, dienochlor, and endosulfan; organotin acaricides such as azocyclotin, cyhexatin, and fenbutatin oxide; pyrazoie acaricides such as acetoprole, fipronil and analogues and derivatives thereof, tebufenpyrad, and vaniliprole; pyrethroid acaricides including: pyrethroid ester acaricides like acrinathrin, bifenthrin, cyhalothrin, cypermethrin, alpha-cypermethrin, fenpropathrin, fenvalerate, flucythrinate, flume-thrin, fluvalinate, tau-fluvalinate, and permethrin, and pyrethroid ether acaricides like halfenprox; quinoxaiine acaricides such as chinomethionat and thioquinox; sulfite ester acaricides such as propargite; tetron is acid acaricides such as spirodiclofen; and form unclassified acaricides such acequinocyl, amidoflumet, arsenous oxide, chloromethiuron, closantel, crotamiton, diafen-thiuron, dichlofluanid, disulfiram, fenazaflor, fenazaquin, fen pyroxi mate, fluacrypyrim, fluenetil, mesulfen, MNAF, nifluridide, pyridaben, pyrimidifen, sulfiram, sulfluramid, sulfur and triarathene.

Suitable insecticides can be selected from a variety of well-known different chemical classes such as chlorinated hydrocarbons, organophosphates, carbamates, pyreth raids, formamidines, borates, phenylpyrazoles, and macrocyclic lactones. Prominent insecticides include imidacloprid, fenthion, fipronil, allethrin, resmethrin, fenvalerate, permetrin, malathion and derivatives thereof. According to one embodiment insecticides are those of the neonicotinoid class, for example acetamiprid, clothianidin, dinotefuran, imidacloprid (mentioned above), nitenpyram, thiacloprid and thiamethoxam. Widely used insect growth regulators (IGRs) include, for example benzoylphenylureas such as diflubenzuron, lufenuron, noviflumuron, hexaflumuron, triflumuron, and tefiubenzuron or substances like fenoxycarb, pyriproxifen, methoprene, kinoprene, hydroprene, cyromazine, buprofezin, pymetrozine and derivatives thereof.

Suitable anthelmintics can be selected from endo-parasiticides and endecticides including groups such as macrocyclic lactones, benzimidazoles, pro-benzimidazoles, imidazothiazoles, tetrahydropyrimidines, organophosphates, piperazines, salicylanilide, and cyclic depsipeptides.

Suitable anthelmintics include broad spectrum macrocyclic lactones, such as avermectins, milbemycins and derivatives thereof, including ivermectin, doramectin, moxidectin, selamectin, emamectin, eprinomectin, milbemectin, abamectin, milbemycin oxime, nemadectin, and derivatives thereof, in free form or in the form of a pharmaceutically acceptable salt. Benzimidazoles, benzimidazole carbamate and pro-benzimidazoles include potent compounds such as thiabendazole, mebendazole, fenbendazole, oxfendazole, oxibendazole, albendazole, luxabendazole, netobimin, parbendazole, flubendazole, cyclobendazole, febantel, thiophanate and derivatives thereof. Imidazothiazoles include highly active compounds such as tetramisole, levamisole, and derivatives thereof. Tetrahydropyrimidines include highly active compounds such as morantel, pyrantel, and derivatives thereof. Organophosphates include potent compounds such as dichlorvos, haloxon, trichlorfon, and derivatives thereof. Salicylanilides include highly active compounds such as closantel, tribromsalan, dibromsalan, oxychlozanide, clioxanide, rafoxanide, brotianide, bromoxanide and derivatives thereof. Cyclic depsipeptides include compounds consisting of amino acids and hydroxycarboxylic acids as ring structural units and 6 to 30 ring atoms, such as PF 1022A, emodepside, and others described in U.S. Pat. No. 6,159,932, which is incorporated herein by reference for all relevant purposes.

Suitable antimicrobial active ingredients include various penicillins, tetracyclines, sulfonamides, cephalosporins, cephamycins, aminoglucosids, trimethoprim, dimetridazoles, erythromycin, framycetin, fruazolidone, various pleuromutilins such as thiamulin, valnemulin, various macrolides, streptomycin, clopidol, salinomycin, monensin, halofuginone, narasin, robenidine, quinolones, etc. Quinolones, preferably fluoroquinolones, include compounds such as those disclosed in U.S. Pat. Nos. 4,670,444; 4,472,405; 4,730,000; 4,861,779; 4,382,892; and 4,704,459; which are incorporated herein by reference. Specific examples of fluoroquinolones include benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, perfloxacin, temafloxacin, tosufloxacin, sarafloxacin, and sparfloxacin. As an additional example of an antibacterial fluoroquinolone for use in animals pradofloxacin may be mentioned. Specific examples of other quinolones include pipemidic acid and nalidixic acid.

Other pharmaceutical or nutraceutical agents known in the veterinary arts, such as vitamins and mineral supplements are also suitable active ingredients.

For example, palatable granule compositions of the present invention may comprise as active ingredients one or more nutraceutical agents such as omega 3 fatty acids, omega 6 fatty acids, methylsulfonylmethane, glucosamine HCl, chondroitin sulfate and manganese ascorbate, St. John's Wort, vegetable glycerin, green food products, probiotics, and antioxidants such as vitamins C and E, beta-carotene and selenium, as well as any other vitamin, mineral, or other dietary or nutritional supplement capable of being formulated into a palatable granule composition of the present invention.

If feasible, pharmaceutically acceptable salts of any of the active ingredients may be used in palatable granule compositions disclosed herein. Furthermore, prodrugs of the active ingredient(s) may also be used in palatable granule compositions disclosed herein.

In an aspect, palatable granule compositions of the present invention comprise one or more active ingredients selected from anti-inflammatory agents and parasiticidal (i.e., anthelmintic) agents.

In another aspect, palatable granule compositions of the present invention comprise an active parasiticidal ingredient selected from abamectin, albendazole, clorsulon, closantel, dichlorophene, dimadectin, doramectin, emodepside, enamectin, eprinomectin, febantel, fenbendazole, imidacloprid, ivermectin, latidectin, lepimectin, levamisole, lufenuron, milbemycin oxime, moxidectin, nitroscanate, oxantel, oxibendazole, piperazine, pyrantel, praziquantel, selamectin, spinosad, triclabendazole, and salts and derivatives thereof.

In an aspect, palatable granule compositions of the present invention comprise an active anti-inflammatory ingredient selected carprofen, dexamethasone, ketoprofen, meloxicam, metacam, naproxen, nimeseulide, pentoxyfilline, phenylbutazone, prednisolone, prednisone, robenacoxib, sulfasalazine, tolfenamic acid, and salts and derivatives thereof.

In certain embodiments, palatable granule compositions of the present invention do not comprise as an active ingredient apoquel, sarolaner, afoxolaner, fluralaner, lotilaner, maropitant, acetaminophen, ibuprofen, flurbiprofen, clavamox, naproxen, meloxicam, ketoprofen, phenylpropanolamine, chlorpheniramine maleate, dextromethorphan, diphenhydramine, famotidine, loperamide, ranitidine, cimetidine, astemizole, terfenadine, terfenadine carboxylate, cetirizine, moxidectin, pyrantel, oclacitinib, milbemycin oxime, or a neurokinin (N)-1 inhibitor.

In an aspect, palatable granule compositions of the present invention comprise carprofen as an active ingredient.

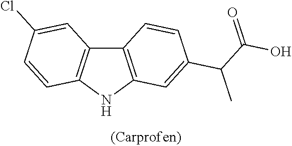 [SMILES: CC(C(=O)O)c1ccc2c(c1)[nH]c1ccc(Cl)cc12]

Carprofen is a non-steroidal anti-inflammatory drug (NSAID) which is marketed under various brand names worldwide. Veterinarians commonly prescribe carprofen as a supportive treatment for various conditions in animals. Carprofen is an especially popular therapeutic for canine and equine administration. Carprofen provides day-to-day treatment for pain and inflammation from various kinds of joint pain, as well as post-operative pain. Carprofen reduces inflammation via inhibition of COX-1 and COX-2. Carprofen's specificity for COX-2 varies from species to species.

In an aspect, palatable granule compositions of the present invention comprise febantel as an active ingredient.

Febantel is an anthelmintic drug useful for de-worming animals and is especially effective against roundworm and tapeworm. Febantel kills parasitic worms by binding to tubulin subunits and interfering with microtubule formation.

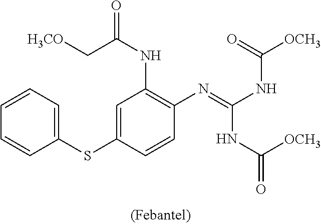 [SMILES: COCC(=O)Nc1cc(Sc2ccccc2)ccc1N=C(NC(=O)OC)NC(=O)OC]

In horses, febantel is readily absorbed from the gastrointestinal tract and is rapidly metabolized to fenbendazole-sulphone, fenbendazole and oxibendazole. Febantel is also absorbed from the intestine in cattle and sheep.

Febantel is also administered to companion animals. For example, in dogs and cats, commercially available Vercom® (a combination of febantel & praziquantel) is unlikely to cause serious adverse effects at typical doses.

In an aspect, palatable granule compositions of the present invention comprise one or more active ingredients in a total amount of 0.001% to 75%, or of 0.005% to 50%, or of 0.01% to 35%, or 0.05% to 20%, or 0.1% to 15%, or 1% to 10%, by weight based on the total weight of the palatable granule composition.

“Disintegrant” means an ingredient, generally not otherwise active, that aids in the break-up of palatable granule compositions of the present invention upon administration to an animal.

In an aspect, palatable granule compositions of the present invention may comprise any pharmaceutically acceptable disintegrant.

In another aspect, palatable granule compositions of the present invention comprise one or more disintegrants selected from agar-agar, potato or tapioca starch, corn starch, pre-gelatinized and modified starches, clays such as bentonite, various silicates, sodium starch glycolate, methyl cellulose, cross-linked sodium carboxymethyl cellulose, microcrystalline cellulose (e.g., Avicel), sodium carbonate, calcium carbonate, hydroxy propylcellulose-low substituted, colloidal silicon dioxide, cellulose polyacrilin potassium (e.g., Amberlite), guar, locust bean, karaya, xanthan, pectin, tragacanth, polyvinylpyrrolidone, crospovidone, rice, carmellose calcium, directly compressible mannitol, and croscarmellose sodium.

In certain embodiments, palatable granule compositions of the present invention do not comprise carboxymethyl cellulose calcium, carboxymethyl cellulose sodium, and/or hydroxypropyl cellulose.

In certain embodiments, palatable granule compositions of the present invention comprise one or more disintegrants selected from crospovidone, sodium starch glycolate, and/or croscarmellose sodium.

Crospovidone (also referred to as cross-linked polyvinyl N-pyrrolidone, or PVP) is a common inactive ingredient in medications and dietary supplements to allow absorption of the active drug. It is considered a synthetic povidone analog. Chemically, crospovidone is an inert and insoluble white to light yellow free-flowing powder. It has hygroscopic, or water-attracting properties with excellent swelling characteristics. It is this swelling characteristic that makes it useful as a disintegrant in pharmaceutical dosage forms. Crospovidone is not absorbed orally.

Sodium starch glycolate is the sodium salt of carboxymethyl ether. Starch glycolates are of rice, potato, wheat or corn origin. Sodium starch glycoate is a white to off-white, tasteless, odorless, relatively free flowing powder, which is used as a pharmaceutical acceptable dissolution excipient for tablet and capsule dosage forms. Sodium starch glycolate absorbs water rapidly, resulting in swelling which leads to rapid disintegration of tablets and granules.

Croscarmellose sodium is an internally cross-linked sodium carboxymethylcellulose for use as a disintegrant in pharmaceutical formulations. The cross-linking reduces water solubility while still allowing the material to swell and absorb many times its weight in water. As a result, it provides superior drug dissolution and disintegration characteristics, thus improving bioavailability by bringing active ingredients into better contact with bodily fluids.

In an aspect, palatable granule compositions of the present invention comprise one or more disintegrants in a total amount by weight of 0% to 60%, or 0.01% to 50%, or 0.1% to 35%, or 1% to 25%, based on the total weight of the palatable granule composition.

In certain embodiments, palatable granule compositions of the present invention do not comprise a disintegrant. In an aspect, palatable granule compositions of the present invention which do not comprise a disintegrant nonetheless exhibit superior disintegration rates as compared to existing granule-based veterinary compositions.

In an aspect, formulations of the palatable granule compositions of the present invention may be modified to obtain the desired palatability and/or a desired disintegration time.

“Binder” or “binding agent” means an ingredient, generally otherwise inactive, which adds cohesiveness to the formulation to provide bonding to form a cohesive mass and to ensure a suitable compacted form. Binders are conventionally used in direct compression tablets and are described in Lieberman et. al., Pharmaceutical Dosage Forms, 2 Ed., Vol. 1, pp. 209-214 (1990).

In certain embodiments, palatable granule compositions of the present invention do not comprise any of microcrystalline cellulose, hydroxypropyl cellulose, hydroxypropyl methyl cellulose, ethyl cellulose, methyl cellulose, sodium carboxy methyl cellulose, polyvinylpyrrolidone (PVP), co-povidone, corn starch, potato starch, pregelatinized starch, polyvinylcaprolactam, xylitol, sorbitol, and/or maltitol.

In certain embodiments, palatable granule compositions of the present invention do not comprise a binder.

In certain embodiments, palatable granule compositions of the present invention do not comprise a binder or a disintegrant, or do not comprises either a binder or disintegrant.

It has been found that exclusion of inactive binders and/or disintegrants allows for maximization of palatable components rather than typical pharmaceutical ingredients, which may not taste or smell appealing to animals. Embodiments lacking binders and/or disintegrants thus achieve high palatability and, consequently, animal compliance.

It has further been found that, surprisingly, embodiments lacking binders nonetheless exhibit desired cohesiveness.

“Wetting agent” means an ingredient, generally otherwise inactive, which tends to attract and/or retain moisture in a pharmaceutical composition. In general, inclusion of a wetting agent increases the solubility of active ingredients in a pharmaceutical or veterinary composition. Palatable granule compositions of the present invention may comprise any pharmaceutically acceptable wetting agent or agents.

In an aspect, palatable granule compositions of the present invention comprise one or more wetting agents selected from gums, waxes, e.g., paraffin wax, glycerin, glycerol, glyceryl, glyceryl stearates, glyceryl hexanoates, glycerol monostearate, miglyol (e.g., miglyol 812, miglyol 840), maltitol, sorbitols, malic acid, cetyl alcohol, ethylene glycol, monomethyl ether, ethylene glycol monomethyl ether, ethylene glycol monoethyl ether, diethylene glycol, diethylene glycol monoethyl ether, triethylene glycol monoethyl ether, diethylene glycol monomethyl ether, triethylene glycol monomethyl ether, methanol, ethanol, isopropanol, methoxy propanol, diethylene glycol monobutyl ether, tetraethylene glycol, triethylene glycol, butyl diglycol, dimethylacetamide, dimethylformamide, n-methylformamide, dipropylene glycol n-butyl ether, diethylene monobutylether acetate, diethylene monoethylether acetate, monomethylacetamide, 2-pyrrolidone, and N-methyl pyrrolidone, propylene glycol, methoxypropanol, polyethylene glycol (“PEG”) of various grades, e.g., PEG 6000, PEG4000, PEG3350, PEG2000, PEG1000, PEG400 and/or PEG300, dipropyleneglycol monomethyl ether, tetrahydrofurfuryl alcohol, Solutol HS 15 (polyglycol mono- and di-esters of 12-hydroxystearic acid), glyceryl cocoate, methoxypolyethylene glycols, polypropylene glycols, polybutylene glycols, tetraglycol, dipropylene glycol n-butyl ether, caprylic/capric glycerides, caprylic glycerides, dibutyl adipate, liquid polyoxyethylene glycols, propylene carbonate, butylene carbonate, solketal, xylene, dimethyl isosorbide, short-, medium- and long chain, and aromatic fatty acids (e.g., butyric acid, capric acid, succinic acid, adipic, sebacic, capriylic acid, lauric acid, myristic acid, strearic acid, linoleic acid, and benzoic acid), glyceryl monooleate, glyceryl ricinoleate, isopropyl myristate, ethyl oleate, ethyl laurate, propylene glycol monocaprylate, propylene glycol monolaurate, spider esters, dibutyl sebacate, triglycerides such as castor oil, cottonseed oil, sesame oil, linseed oil, safflower oil, peanut oil, soybean oil, coconut oil, olive oil, corn oil, and almond oil, silicones, hyaluronic acid, honey, molasses, aloe, lecithin, panthenol, alginate, polysorbate 80, Span 80 (sorbitan monooleate), and other surfactants, emulsifiers, synthetic alcohols (e.g., hydroxystearate, myristate, oleate), sucrose, triacetin, water, and/or mineral oils.

In certain embodiments, palatable granule compositions of the present invention do not comprise any of miglyol, Solutol HS 15 (polyglycol mono- and di-esters of 12-hydroxystearic acid), ethanol, or triglycerides (e.g., castor oil, cottonseed oil, sesame oil, safflower oil, peanut oil, soybean oil, coconut oil, and/or olive oil).

In another aspect, palatable granule compositions of the present invention comprise one or more wetting agents selected from honey, molasses, gums, gelatins, waxes, paraffin wax, 2-pyrrolidone, water, oil, surfactants, emulsifiers, alginate, glycerin, liquid palatants, polysorbate 80, glycerol, propylene glycol, polyethylene glycol (“PEG”) of various grades, e.g., PEG 6000, PEG4000, PEG3350, PEG2000, PEG1000, PEG400 and/or PEG300.

In certain embodiments, palatable granule compositions of the present invention comprise one or more wetting agents which are palatable, such as honey or molasses.

In other embodiments, palatable granule compositions of the present invention do not comprise a palatable wetting agent.

In an aspect, palatable granule compositions of the present invention comprise one or more wetting agents in an amount of 5% to 80%, or 15% to 70%, or 30% to 60%, based on the total weight of the palatable granule composition.

“Stiffening agent” or “stiffener” means an inactive ingredient, which is not a binder or binding agent, which is solid or highly viscous at room temperature and, generally, can be melted with heat and solidify or become viscous at room temperature to provide a stiffened structure. Palatable granule compositions of the present invention may optionally comprise any pharmaceutically acceptable stiffening agent.

In an aspect, palatable granule compositions of the present invention comprise one or more stiffening agents selected from microcrystalline cellulose, hydroxypropyl cellulose, hydroxypropyl methyl cellulose, ethyl cellulose, polyvinylpyrrolidone, co-povidone, acacia, tragacanth gum, gelatin, sucrose, lactose (e.g., hydrous, anhydrous, monohydrate), xylitol, sorbitol, maltitol, corn starch, potato starch, alginate, waxes, solid lipids, and polyethylene glycol (“PEG”) of various grades, e.g., PEG 6000, PEG4000, PEG3350, PEG2000, PEG1000, PEG400, PEG300 (e.g., PEG300 or higher, generally).

In another aspect, palatable granule compositions of the present invention comprise one or more stiffening agents which also act as wetting agents selected from waxes (e.g., paraffin wax), solid lipids, and polyethylene glycol (“PEG”) of various grades, e.g., PEG 6000, PEG4000, PEG3350, PEG2000, PEG1000, PEG400 and/or PEG300 (e.g., PEG 300 or higher, generally).

In certain embodiments, palatable granule compositions of the present invention do not comprise stiffeners which may also act as binding agents, e.g., microcrystalline cellulose, hydroxypropyl cellulose, hydroxypropyl methyl cellulose, ethyl cellulose, methyl cellulose, sodium carboxy methyl cellulose, polyvinylpyrrolidone (PVP), co-povidone, corn starch, potato starch, pregelatinized starch, polyvinylcaprolactam, xylitol, sorbitol, maltitol.

In certain embodiments, palatable granule compositions of the present invention do not comprise microcrystalline cellulose, hydroxypropyl cellulose, hydroxypropyl methyl cellulose, ethyl cellulose, polyvinylpyrrolidone, co-povidone.

In an aspect, palatable granule compositions of the present invention comprise one or more stiffening agents in an amount of 1% to 75%, or 5% to 50%, or 10% to 30% based on the total weight of the palatable granule composition.

In certain embodiments, palatable granule compositions of the present invention do not comprise a stiffening agent.

In an embodiment, palatable granule compositions of the present invention contain starch.

In another embodiment, palatable granule compositions of the present invention do not contain starch as a binder.

In yet another embodiment, palatable granule compositions of the present invention do not contain any starch.

In an aspect, the present disclosure provides for palatable granule compositions which contain water. In an aspect, palatable granule compositions of the present invention may comprise 0% to 20% water, or 0.0001% to 10% water, or 0.001% to 5% water, or 0.01% to 2% water, based on the total weight of the palatable granule composition.

In another aspect, the present disclosure further provides for palatable granule compositions which are substantially free of water.

As used herein, the terms “treat,” “treating,” “treatment” and grammatical variations thereof mean subjecting an animal subject to a protocol, regimen, process or remedy, in which it is desired to obtain a physiologic response or outcome in that subject. In particular, the methods and compositions of the present invention may be used to slow the development of disease symptoms or delay the onset of the disease or condition or halt the progression of disease development. However, because every treated animal subject may not respond to a particular treatment protocol, regimen, process or remedy, treating does not require that the desired physiologic response or outcome be achieved in each and every subject or subject population. Accordingly, a given subject or subject population may fail to respond or respond inadequately to treatment.

As used herein, the terms “ameliorate”, “ameliorating” and grammatical variations thereof mean to decrease the severity of the symptoms of a disease in a subject.

As used herein, the terms “prevent”, “preventing” and grammatical variations thereof mean to administer a compound or composition of the present invention to a subject animal which has not been diagnosed as having the disease or condition at the time of administration, but which could be expected to develop the disease or condition or be at increased risk for the disease or condition. Preventing also includes administration of at least one compound or a composition of the present invention to those subjects thought to be predisposed to the disease or condition due to age, familial history, genetic or chromosomal abnormalities, due to the presence of one or more biological markers for the disease or condition and/or due to environmental factors.

In an aspect, the present disclosure provides for a method of treating an animal comprising administering to the animal a palatable granule composition described herein.

In an aspect, the palatable granule composition may be administered to an animal one, two, three, four, five, six, seven, eight, nine, or ten times daily, depending on the dosage, disease or condition severity, and the particular animal species and size.

In an aspect, the palatable granule composition may be administered in a dosage of one, two, three, four, five, six, seven, eight, nine, or ten palatable granules, depending on the disease or condition severity and the particular animal species and size.

In an aspect, the palatable granule composition may be administered to an animal to be treated.

In an aspect, the animal to be treated is a dog, a cat, a horse, a pig, a sheep, a goat, a cow, a rabbit, a llama, a deer, an elk, or poultry.

In another aspect, the animal to be treated is a dog, a cat, or a horse.

Palatable granule compositions of the present invention may, optionally, contain additional ingredients and/or materials commonly used in such veterinary compositions. In other embodiments, the optional ingredients are not present. These ingredients and materials are well known in the art and include (1) fillers or extenders, such as starches, lactose, sucrose, glucose, mannitol, and silicic acid; (2) solution retarding agents, such as paraffin; (3) absorption accelerators, such as quaternary ammonium compounds; (4) lubricants, such as sodium oleate, sodium stearate, calcium stearate, zinc stearate, magnesium stearate, polyethylene glycol, talc, mineral oil, stearic acid, sodium benzoate, sodium acetate, sodium chloride, and sodium lauryl sulfate; (5) suspending agents, such as ethoxylated isostearyl alcohols, polyoxyethylene sorbitol and sorbitan esters, microcrystalline cellulose, aluminum metahydroxide, bentonite, agar-agar and tragacanth; (6) buffering agents, such as potassium metaphosphate, potassium phosphate, monobasic sodium acetate and sodium citrate anhydrous and dihydrate; (7) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (8) inert diluents, such as dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, starch, and water or other solvents; (9) preservatives, such as Nipagin, Nipasol, alcohol, antimicrobial agents, benzoic acid, sodium benzoate, benzyl alcohol, sorbic acid, parabens, and isopropyl alcohol; (10) surface-active agents; (11) dispersing agents, such as synthetic and natural gums including tragacanth, acacia, alginate, dextran, sodium carboxymethylcellulose, methylcellulose, polyvinylpyrrolidone and gelatin; (12) control-release or absorption-delaying agents, such as hydroxypropylmethyl cellulose, other polymer matrices, biodegradable polymers, liposomes, microspheres, aluminum monosterate, gelatin, and waxes; (13) opacifying agents; (14) adjuvants; (15) emulsifying and suspending agents; (16), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (17) antioxidants, such as ascorbic acid, ascorbyl palmitate, butylated hydroxyanisole, butylated hydroxytoluene, hypophophorous acid, monothioglycerol, propyl gallate, sodium ascorbate, sodium bisulfate, sodium formaldehyde sulfoxylate and sodium metabisulfite; (18) agents which render the formulation isotonic with the blood of the intended recipient, such as sugars and sodium chloride; (19) thickening agents; (20) coating materials, such as lecithin; and (21) sweetening, coloring, perfuming and preservative agents.

Each such ingredient or material must be pharmaceutically acceptable in the sense of being compatible with the other ingredients of the formulation and not injurious to the subject animal.

Palatable granule compositions of the present invention may be manufactured by any method, such as by single pot granulation, fluid bed top spray granulation, high sheer granulation/fluid bed drying combination, continuous fluid bed granulation, spray drying, and other methods. Dry compaction (i.e., dry granulation) and wet extrusion followed by sizing and drying may also be employed.

The following examples serve to illustrate certain aspects of the disclosure and are not intended to limit the disclosure.

### EXAMPLES

### Example 1—Exemplary Palatable Granule Placebo Formulations

Table 1 sets forth exemplary formulations of palatable granule compositions of the present invention which comprise two ingredients, namely a solid palatant and PEG3350 as a wetting agent, in various amounts.

TABLE 1

| | Ex. 1 | Ex. 2 | Ex. 3 | Ex. 4 | Ex. 5 |
|---|---|---|---|---|---|
| | Weight, | Weight, | Weight, | Weight, | Weight, |
| Ingredient | % | % | % | % | % |
| Blueberry Powder | 46.4 | 0.0 | 0.0 | 0.0 | 0.0 |
| Apple Powder | 0.0 | 59.1 | 0.0 | 0.0 | 0.0 |
| Pumpkin Powder | 0.0 | 0.0 | 65.0 | 0.0 | 0.0 |
| Sweet Potato | 0.0 | 0.0 | 0.0 | 65.0 | 0.0 |
| Powder | | | | | |
| Solid Liver | 0.0 | 0.0 | 0.0 | 0.0 | 54.2 |
| Palatant | | | | | |
| PEG3350 | 53.6 | 40.9 | 35.0 | 35.0 | 45.8 |

### Example 2—Exemplary Palatable Granule Placebo Formulations

Table 2 sets forth further exemplary formulations of palatable granule compositions of the present invention which comprise two ingredients, namely a solid palatant and either a polyethylene glycol, paraffin wax or Span 80 (sorbitan monooleate) as a wetting agent, in various amounts.

TABLE 2

| | Ex. 6 | Ex. 7 | Ex. 8 | Ex. 9 | Ex. 10 | Ex. 11 | Ex. 12 |
|---|---|---|---|---|---|---|---|
| Ingredient | Weight, % | Weight, % | Weight, % | Weight, % | Weight, % | Weight, % | Weight, % |
| Solid Liver | 61.9 | 67.2 | 74.3 | 65.3 | 76.7 | 68.6 | 72.9 |
| Palatant | | | | | | | |
| PEG2000 | 38.1 | 0.0 | 0.0 | 0.0 | 0.0 | 0.0 | 0.0 |
| PEG1000 | 0.0 | 32.8 | 0.0 | 0.0 | 0.0 | 0.0 | 0.0 |
| PEG300 | 0.0 | 0.0 | 25.7 | 0.0 | 0.0 | 0.0 | 0.0 |
| Paraffin wax | 0.0 | 0.0 | 0.0 | 34.7 | 0.0 | 0.0 | 0.0 |
| Span 80 | 0.0 | 0.0 | 0.0 | 0.0 | 23.3 | 0.0 | 0.0 |
| Glycerol | 0.0 | 0.0 | 0.0 | 0.0 | 0.0 | 31.4 | 0.0 |
| Soybean Oil | 0.0 | 0.0 | 0.0 | 0.0 | 0.0 | 0.0 | 27.1 |

Example formulations Ex. 6, Ex. 7, Ex. 8, and Ex. 9 retained greater than 90% mass upon sieving.

No size data was obtained for the remaining example formulations.

### Example 3—Exemplary Drug-Loaded Palatable Granule Formulations

Table 3 sets forth further exemplary formulations of palatable granule compositions of the present invention which comprise an active ingredient, a solid palatant, and a wetting agent in various amounts.

TABLE 3

| | Ex. 13 | Ex. 14 | Ex. 15 |
|---|---|---|---|
| Ingredient | Weight, % | Weight, % | Weight, % |
| Carprofen Active | 5.0 | 5.0 | 33.3 |
| Solid Liver Palatant | 65.0 | 61.0 | 0.0 |
| Blueberry powder | 0.0 | 0.0 | 47.4 |
| Crospovidone | 0.0 | 4.0 | 0.0 |
| PEG3350 | 30.0 | 30.0 | 0.0 |
| PEG1000 | 0.0 | 0.0 | 19.3 |

Exemplary formulations Ex. 13 and Ex. 14 differ in that Ex. 14 further comprises crospovidone, a disintegrant. Ex. 15 comprises a high level of drug load at 33.3%.

It has surprisingly been found that formulations according to the present invention are capable of carrying both a high active drug load and a high palatant load.

Each of Ex. 13, Ex. 14 and Ex. 15 comprises a high concentration of palatant ranging from 47.4% to 65.0%, which would achieve high animal compliance and thus allow for administration of active ingredients which may be comprised at high concentrations as well in palatable granules of the present invention, e.g. Ex. 15.

Successful animal compliance achieved with palatable granules of the present invention is set forth in Example 4 below.

### Example 4—Palatability Acceptance Results

A palatability acceptance study was conducted with 11 mixed-breed dogs for two consecutive days of offering in a dog bowl the placebo granules as set forth in Table 4 below.

Dogs were allowed to voluntarily consume the granules from the dog bowl and full voluntary consumption was recorded. On both days, 9 out of 11 dogs fully consumed the granules provided.

Table 4 sets forth the two-ingredient formula for the placebo used in the palatability study

TABLE 4
Palatability: ≥80% full voluntary consumption

| Ingredient | Weight, % |
|---|---|
| Solid Liver Palatant | 55.0 |
| PEG3350 | 45.0 |

1. A palatable granule composition comprising:
(a) at least one palatant;
(b) at least one wetting agent; and
(c) at least one active ingredient.

2. The palatable granule composition of claim 1, wherein the (a) at least one palatant is selected from the group consisting of apple powder, bean powder, beet powder, pepper powder, blueberry powder, broccoli powder, squash powder, cabbage powder, carrot powder, cauliflower powder, celery powder, chevril powder, chive powder, corn powder, cranberry powder, dill powder, kale powder, leek powder, lemon powder, mushroom powder, onion powder, orange powder, potato powder, pea powder, pumpkin powder, shallot powder, spinach powder, tomato powder, tomatillo powder, sweet potato powder, zucchini powder, natural and artificial meat powders such as liver powder and artificial beef, yeast, tapioca syrup, honey, and salt.

3. The palatable granule composition of claim 1, wherein the (b) at least one wetting agent is selected from the group consisting of glycerol, glycerol monostearate, maltitol, sorbitols, malic acid, cetyl alcohol, ethylene glycol, ethylene glycol monoethyl ether, diethylene glycol, diethylene glycol monoethyl ether, diethylene glycol monomethyl ether, methanol, ethanol, isopropanol, methoxy propanol, diethylene glycol monobutyl ether, tetraethylene glycol, triethylene glycol, butyl diglycol, dimethylacetamide, dimethylformamide, n-methylformamide, dipropylene glycol n-butyl ether, propylene glycol, PEG 6000, PEG4000, PEG3350, PEG2000, PEG1000, PEG400, PEG300, and mineral oil.

4. The palatable granule composition of claim 1, further comprising (d) at least one stiffening agent selected from the group consisting of microcrystalline cellulose, hydroxypropyl cellulose, hydroxypropyl methyl cellulose, ethyl cellulose, polyvinylpyrrolidone, co-povidone, acacia, tragacanth gum, gelatin, sucrose, lactose, xylitol, sorbitol, maltitol, corn starch, potato starch, alginate, waxes, solid lipids, PEG 6000, PEG4000, PEG3350, PEG2000, PEG1000, PEG400 and PEG300.

5. The palatable granule composition of claim 1, further comprising (e) at least one disintegrant selected from the group consisting of agar-agar, potato starch, tapioca starch, corn starch, pre-gelatinized and modified starches, clays, alginates, alginic acid, silicates, sodium starch glycolate, methyl cellulose, cross-linked sodium carboxymethyl cellulose, microcrystalline cellulose, sodium carbonate, calcium carbonate, hydroxy propylcellulose-low substituted, colloidal silicon dioxide, cellulose polyacrilin potassium, gums, guar, locust bean, karaya, xanthan, pectin, tragacanth, polyvinylpyrrolidone, crospovidone, carmellose calcium, directly compressible mannitol, and croscarmellose sodium.

6. The palatable granule composition of claim 1, wherein the (c) at least one active ingredient is selected from the group consisting of anti-parasitic agents, acaricidic agents, anthelmintic agents, insecticidal agents, antimicrobial agents, antiviral agents, antibiotic agents, anti-inflammatory agents, psychotropic agents, proton pump inhibitors, pain relievers, anti-allergy medications, and antihypertensives.

7. The palatable granule composition of claim 1, wherein the (a) at least one palatant is present in an amount of 1% to 90%, based on the total weight of the palatable granule composition.

8. The palatable granule composition of claim 1, wherein the (a) at least one palatant is present in an amount of 10% to 80%, based on the total weight of the palatable granule composition.

9. The palatable granule composition of claim 1, wherein the (b) at least one wetting agent is present in an amount of 5% to 80%, based on the total weight of the palatable granule composition.

10. The palatable granule composition of claim 4, wherein the (d) at least one stiffening agent is present in an amount of 1% to 75%, based on the total weight of the palatable granule composition.

11. The palatable granule composition of claim 5, wherein the (e) at least one disintegrant is present in an amount of 0.01% to 50%, based on the total weight of the palatable granule composition.

12. The palatable granule composition of claim 1, wherein the (c) at least one active ingredient is present in an amount of 0.001% to 75%, based on the total weight of the palatable granule composition.

13. The palatable granule composition of claim 12, wherein the (c) at least one active ingredient is present in an amount of 0.005% to 50%, based on the total weight of the palatable granule composition.

14. The palatable granule composition of claim 1, wherein the palatable granule composition is manufactured by low shear wet granulation, high shear wet granulation, or mortar and pestle.

15. A method of treating an animal with a disease or condition comprising administering to said animal a palatable granule composition of claim 1, wherein the disease or condition is inflammation or a parasite.

16. The method of claim 15, wherein said palatable granule composition is not administered in combination with or as part of any other dosage form or food.

17. The method claim 15, wherein the animal is a dog, a cat, a horse, a pig, a llama, a rabbit, a goat, a sheep, a deer, an elk, a cow, or poultry.

18. The method of claim 17, wherein the animal is a dog or a cat.

19. The method claim 15, wherein the disease or condition is inflammation.

20. The method claim 15, wherein the disease or condition is a parasite.
